(19) United States
(12) Patent Application Publication
(10) Pub. No.: US 2026/0078325 A1
(43) Pub. Date: Mar. 19, 2026

(54) ACCOMMODATION DEVICE FOR A CULTURE MEDIUM CARRIER

(71) Applicant: Pharmabotix AG, Seengen (CH)

(72) Inventor: Luca GLANZMANN, St. Erhard (CH)

(73) Assignee: Pharmabotix AG, Seengen (CH)

(21) Appl. No.: 19/105,094

(22) PCT Filed: Aug. 10, 2023
(86) PCT No.: PCT/EP2023/072218

§ 371 Date: Feb. 20, 2025

(30) Foreign Application Priority Data
Aug. 22, 2022 (EP) 22191545.7

(51) Int. Cl. C12M 1/22; C12M 1/00; C12M 1/12; C12M 1/36

(52) U.S. Cl. CPC C12M 23/10; C12M 23/38; C12M 37/06; C12M 41/48

(57) ABSTRACT

An accommodation device for a culture medium carrier, such as a petri dish in particular, is specified. The accommodation device has a hermetically closable interior for accommodation of the culture medium carrier. By a wirelessly remote-controllable drive, the accommodation device can be opened and closed, in order respectively to provide access to the culture medium carrier and to hermetically close the interior with the culture medium carrier accommodated therein. The accommodation device as a whole may be introduced into and removed from a hermetically insulable space in an insulator. Also specified is a method of identifying microbes in the atmosphere of an insulator.

### TECHNICAL FIELD

The present invention relates to an accommodation device for a culture medium carrier, such as a petri dish in particular. The accommodation device is configured to be introduced into a hermetically closable insulator in order to identify microbes in the atmosphere of the insulator. The invention therefore further relates to a method for identifying microbes in the atmosphere of an insulator.

### PRIOR ART

Aseptic insulators are used, in particular, in the pharmaceutical industry in order to generate a defined microbe-free atmosphere relative to the surrounding work space, so that sensitive or dangerous products can be processed. Insulators are often used, for example, as part of filling systems which serve for filling containers, such as for example vials, ampoules or syringes, with a bio-pharmaceutical product. Due to the highly pure and in particular microbe-free atmosphere in the insulator, the risk of a contamination of the product by microbes, such as for example viruses or bacteria, can be significantly reduced or in the best case even substantially eliminated during the filling process. The containers are hermetically closed while still in the insulator before they are removed from the insulator for the purpose of storage, transport and distribution. The sterility of the products accommodated in the closed container can be ensured thereby to a high degree.

The atmosphere in such insulators is generally monitored during the course of so-called bio-monitoring, so as always to be able to ensure a microbiological contamination of the insulator space, even during long-term use of the insulator. To this end, for example, petri dishes which carry a suitable culture medium are positioned in the insulator space. The culture medium is exposed thereby to the atmosphere in the insulator space. Any microorganisms present in the insulator atmosphere settle in the petri dish and grow therein to form detectable colonies.

In the prior art, the handling of the petri dishes, in particular the positioning and the opening and reclosing with a lid, is generally carried out in the interior of the insulator by means of a glove port. With the flexibly designed glove feedthroughs, there is the risk of damage and resulting leakage. Microbes which settle in the culture medium and which potentially have already multiplied therein can also be transmitted when handled by means of the glove port, and as a result spread in the insulator.

It is therefore known to provide in the interior of the insulator a robot with gripper arms which takes over the handling of the culture medium carrier, i.e. the petri dishes, so that the glove port can be dispensed with.

For example, a packaging system for filling and closing medicaments in containers which comprises a 6-axis robot for handling petri dishes is disclosed in WO 2020/233854 A1.

WO 2021/156263 A1 discloses a method for the automated monitoring of microbes in an insulator. The transfer of the culture medium carrier from a transfer lock to a respectively provided culture medium carrier holder is carried out with the assistance of a robot.

Robots, in particular multi-joint robots and robots with gripper arms, however, are generally complex in terms of construction and as a result expensive. The control or programing of robots is also generally difficult and only possible by trained specialist personnel. An adaptation of simple parameters, such as for example a different positioning of the petri dishes, is thus often not possible in a simple manner. Moreover, the retrofitting of existing insulators with a robot is complex or often not even possible at all.

### SUMMARY OF THE INVENTION

It is therefore an object of the invention to specify a device which permits a simple and safe handling of culture medium carriers in an insulator. The device should have the simplest possible construction and if possible also be able to be used without much effort even in existing insulators.

An accommodation device for a culture medium carrier, such as in particular a petri dish, is proposed in order to achieve this object, as specified in claim 1. Moreover, a method for identifying microbes in the atmosphere of an insulator is specified in claim 11.

Advantageous embodiments of the invention are laid down in the dependent claims.

The present invention thus provides an accommodation device for a culture medium carrier, such as a petri dish in particular, comprising
a hermetically closable interior for accommodation of the culture medium carrier; and
a wirelessly remote-controllable drive for opening and closing the accommodation device in order respectively to provide access to the culture medium carrier or to hermetically close the interior with the culture medium carrier accommodated therein.

The accommodation device as a whole can be introduced into and removed from a hermetically insulable space in an insulator.

Since the accommodation device comprises a wirelessly remote-controllable drive for opening and closing and can be introduced as a whole into the insulator with the culture medium carrier accommodated therein, no further handling of the culture medium carrier is required in the interior of the hermetically insulated insulator space, for example by means of a glove port or by means of robot arms. As a result, the handling can take place exclusively by means of a wireless remote control in a safe and very simple manner. To this end, the culture medium carrier is inserted into the accommodation device outside the insulator in a protective atmosphere, the accommodation device then being hermetically closed. The accommodation device can then be introduced into the insulator space, where it can be sterilized from outside in the still closed state without the growing medium or culture medium being damaged during the decontamination. When the insulator is ready for production, the drive of the accommodation device can be activated by means of the remote control in order to open the accommodation device and to provide access to the culture medium carrier. The culture medium is exposed thereby to the atmosphere of the insulator space. After a certain exposure time, the accommodation device can be closed again by means of the remote control so that no microbes can pass outwardly from the culture medium carrier and in the reverse direction. In this state, the accommodation device can be taken out from the insulator together with the culture medium carrier which is accommodated therein and hermetically insulated from the environment. The culture medium carrier can then be removed from the accommodation device and transported further for incubation and/or microorganism detection. Alternatively, it can also be transported further together with the accommodation device.

As the accommodation device as a whole can be introduced into and removed from the insulator together with the culture medium carrier, it is also particularly well suited for use in existing insulators or for retrofitting. The field of application is, in particular, in so-called “gloveless” insulators, i.e. insulators which do not have any manipulating gloves. Moreover, in comparison with a typical robot arm, the accommodation device is not only able to be operated and controlled more easily but typically can also be constructed significantly more simply and thereby generally more cost-effectively as a whole.

The culture medium carrier can be a petri dish, in particular. The culture medium is then accommodated in the interior of the petri dish, wherein the petri dish, in particular, can have a lid. Preferably, the culture medium carrier comprises a dish and a lid, wherein the lid can be advantageously locked to the dish. As a result, the culture medium carrier can be transported safely outside the accommodation device and, in particular, such that the culture medium contained therein is protected from the environment. However, it is also completely possible that the culture medium carrier comprises a dish and a lid which cannot be locked to one another. In principle, however, it is also conceivable that the culture medium carrier is a simple plate which carries the culture medium.

A flat, round, generally transparent dish, which may or may not have a lid, is regarded as a petri dish. If the petri dish comprises a lid, this can engage over or below the dish, i.e. the lid edge can extend in the radial direction beyond the dish or be arranged entirely inside the dish.

The culture medium carrier, in particular the petri dish, can form a part of the accommodation device and be distributed together, for example.

When the accommodation device is closed, the interior of the accommodation device is thus hermetically insulated from the environment. Any microbes which are present thus cannot pass into the interior or pass therefrom to the outside. If a culture medium carrier is accommodated in the interior of the closed accommodation device, it is also hermetically insulated from the environment.

The drive serves for opening and closing the accommodation device, i.e. for opening up or hermetically insulating the interior. The drive preferably is a motor, in particular an electric motor. The remote control of the drive can take place, for example, via infrared, radio, Wi-Fi, or WLAN or Bluetooth. To this end, the accommodation device preferably comprises a remote control which is correspondingly configured and, depending on the case, specifically provided therefor. The remote control can be, however, a smartphone on which an app for operating the accommodation device is installed.

An “insulator” is generally understood to be a container, the interior thereof being able to be hermetically insulated, i.e. separated, relative to a surrounding work space. A defined atmosphere for processing sensitive or dangerous products, such as in particular pharmaceutical products, can be generated in the interior. The insulator can be a stationary or mobile insulator. The insulator generally stands in a room of a building which forms a work space surrounding the insulator. This is in contrast to a clean room, for example, of a laboratory or another building which itself forms the work space. The insulator can form, in particular, a part of a filling system which, for example, serves for filling containers with a pharmaceutical product.

The accommodation device preferably also comprises at least one energy storage element, such as in particular a battery, for supplying electrical energy to the drive. The battery can be a rechargeable or a single-use battery. The presence of an energy storage element makes the handling of the accommodation device particularly simple when introduced into or removed from the insulator.

In a particularly preferred embodiment, the accommodation device is configured, when opened, to lift a lid of the culture medium carrier off a dish of the culture medium carrier. As a result, the culture medium carrier can be closed outside the insulator, inserted into the accommodation device, and only opened in the hermetically insulated space of the insulator. Preferably, the accommodation device is also configured, when closed, to position the lid back onto the dish. As a result, the culture medium carrier can be closed again in the insulator.

Preferably, the accommodation device comprises a first holding element for securing the lid of the culture medium carrier. The first holding element can serve, in particular, for opening and/or closing the culture medium carrier when the accommodation device is opened or closed. Moreover, the accommodation device preferably comprises a second holding element for securing the dish of the culture medium carrier. The second holding element can serve, in particular, for opening and/or closing the culture medium carrier when the accommodation device is opened or closed. If both the first holding element and the second holding element are present, the culture medium carrier can be opened and closed again particularly safely.

The first holding element, in particular, is preferably one or more clamping elements which clamp the lid of the culture medium carrier preferably from one or more radial directions. Even if this is not preferred, the first holding element could alternatively be formed by a fixing element which bears from below against the lid, i.e. bears against the lid in the direction in which it is lifted off the dish when opened.

The second holding element is preferably formed by a fixing element which is positioned from above on the dish, i.e. is positioned on the dish counter to the direction in which the closure lid is lifted off the dish when opened. The fixing element, which can be configured in particular as a fixing ring, is preferably positioned on the outer radial edge of the dish. Preferably, the fixing element also clamps the dish so that when the interior of the accommodation device is closed the dish cannot be rotated relative to the fixing element. Such a clamping of the dish can serve, in particular, to permit a rotation of the closure lid relative to the dish, in order to unlock or to lock the culture medium carrier by means of a bayonet or threaded closure. In principle, however, it is also conceivable that the second holding element, as in the above-mentioned preferred variant of the first holding element, is one or more clamping elements which preferably clamp the dish of the culture medium carrier from one or more radial directions.

The culture medium carrier preferably comprises a dish which can be closed by a lid, as mentioned above. An interior which serves for accommodating a culture medium is then preferably defined by the dish and the lid together. The lid can preferably be locked to the dish. This enables a safe transport of the culture medium carrier outside the accommodation device. In a particularly preferred embodiment, the accommodation device is configured to unlock and to lock the culture medium carrier which is accommodated in the hermetically closed interior of the accommodation device, i.e. to unlock the lid relative to the dish or to lock the lid thereto, in order to make accessible or to close the interior of the culture medium carrier. As a result, the handling of the culture medium carrier is particularly safe, since it can then be unlocked and opened and closed and locked again in the interior of the hermetically closed insulator.

For the unlocking and locking of the culture medium carrier in the interior of the accommodation device, the accommodation device preferably comprises an actuating element which enables an unlocking and locking of the culture medium carrier by hand. As a result, the accommodation device can be produced and operated particularly simply. The actuating element preferably forms a part of the locking mechanism which is specified further below. In other embodiments, however, it can also be advantageous if the accommodation device comprises an actuating element which is driven by a motor, i.e. enables automatic unlocking and locking of the culture medium carrier.

The culture medium carrier can comprise, in particular, a threaded or bayonet closure. A bayonet closure is a closure in which the two parts to be connected are connected together by being inserted in one another and then rotated in opposing directions. According to a preferred embodiment, in particular, the accommodation device comprises a locking mechanism for unlocking and locking such a culture medium carrier with a threaded or bayonet closure. To this end, the locking mechanism is preferably configured to rotate the lid relative to the dish of the culture medium carrier and, in the case of a bayonet closure, previously to have inserted the lid and the dish into or onto one another. In order to ensure a secure function of the locking mechanism, it preferably comprises one or more slotted guides therefor. The one or more slotted guides advantageously serve to provide a guide for inserting the lid and dish onto or into one another and/or for rotating the lid relative to the dish. Since many commercially obtainable culture medium carriers and, in particular, petri dishes have a threaded or bayonet closure, such an embodiment of the accommodation device is particularly advantageous. A threaded or bayonet closure also permits a particularly simple and yet secure locking of a lid to a dish of a culture medium carrier.

The locking mechanism preferably comprises a first holding element for securing the lid of a culture medium carrier, wherein the first holding element can be moved from an initial position into a pushed-down position and from the pushed-down position into a pushed-down rotated position, in order to lock a culture medium carrier which comprises a bayonet closure. If the accommodation device comprises a second holding element, which then preferably serves for securing the dish of the culture medium carrier in an immovable position when the first holding element is pushed down and rotated.

The locking mechanism is preferably configured to be operated manually by a user for the unlocking and locking. In principle, however, it is also conceivable that a motor is present in order to operate the locking mechanism. The motor may or may not be formed by the already mentioned drive.

The accommodation device preferably comprises a receptacle and a closure lid which together define the interior provided for accommodating the culture medium carrier. Preferably, in the closed state of the accommodation device the interior is defined exclusively by the receptacle and the closure lid. The receptacle and/or the closure lid preferably comprises a sealing element in order to seal the interior hermetically relative to the environment. The sealing element advantageously extends fully circumferentially around the interior. Preferably, the closure lid can be pivoted for opening and closing the interior over an angular range of at least 45°, preferably at least 90°, further preferably even at least 180° about a rotary joint. A pivoting of the closure lid relative to the receptacle by at least 45° or by at least 90° permits a sufficiently large or even complete upward exposure of the part of the interior defined by the receptacle. As a result, the accommodation device is well-suited, in particular, for use in insulators with a vertical air flow. A pivoting by at least 180° permits an optimal air circulation for the exposed receptable from all sides, i.e. the accommodation device is thus not only well suited for use in insulators with a vertical air flow, but also for use in insulators with a horizontal air flow.

Preferably, it is possible to select the angular range over which the closure lid can be pivoted relative to the receptacle about the rotary joint. In other words, it is preferably possible by means of the remote control to adjust the angular range over which the closure lid is pivoted when opened, or it is possible by means of the remote control to stop the pivoting of the closure lid, for example, after a certain angular range. As a result, the culture medium carried by the culture medium carrier can be optimally positioned in the air flow of the insulator and exposed thereto, wherein it can be any horizontal and/or vertical airflow. The accommodation device preferably comprises a control which controls the pivoting of the closure lid by means of the drive, wherein the accommodation device also advantageously has a storage element for storing the angular range about which the closure lid can be pivoted when opened. In this manner, the open position of the closure lid relative to the receptacle can be adapted to the respective application and the conditions which are present in the insulator.

The accommodation device preferably comprises a fully circumferential, at least duplicated, sealing lip in order to achieve and to ensure a particularly good hermetic seal of the interior to the outside in the closed state.

The present invention also relates to a method for identifying and, in particular, for monitoring microbes in the atmosphere of an insulator, wherein the method comprises at least the following steps:
introducing an accommodation device, which is preferably configured as specified above, into a space of the insulator, wherein the accommodation device comprises a hermetically closed interior in which a culture medium carrier, such as in particular a petri dish, is accommodated;
hermetically closing the space of the insulator with the accommodation device accommodated as a whole therein; and
opening the interior of the accommodation device by means of a remote control which is arranged outside the hermetically closed space of the insulator.

The above-mentioned method steps are preferably carried out in the specified sequence. However, the method can have any other method steps.

When opening the interior, the remote control is generally arranged in a work space surrounding the insulator. The operation of the remote control is generally carried out by a user, i.e. a human being. In certain embodiments, the remote control can also have an interface to a computer unit or control unit in order to permit a machine-controlled operation of the remote control.

Following the above-mentioned method steps, the culture medium carrier with the culture medium carried thereby is typically exposed over a certain length of time, which is denoted as the exposure time, to the atmosphere of the hermetically closed space of the insulator. Any microbes potentially present in the atmosphere of the insulator space can settle on the culture medium during this exposure time.

The method can also comprise the following steps which preferably are carried out after the above-mentioned steps:
hermetically closing the interior of the accommodation device by means of the remote control after a certain exposure time, during which a culture medium carried by the culture medium carrier has been exposed to the atmosphere of the hermetically closed space of the insulator;
opening the space of the insulator; and
removing the accommodation device as a whole from the space of the insulator.

For opening the space of the insulator, the insulator comprises preferably an access opening which serves, in particular, for introducing and removing the accommodation device. The access opening can be configured as a lock which permits an introduction or removal of the accommodation device such that, as a result, the atmosphere in the insulator space is not influenced at all, or only to a small extent. The introduction and/or removal of the accommodation device is generally carried out by a user, for example by laboratory personnel, but alternatively it can also be carried out by a machine, such as for example a robot.

The culture medium carrier can comprise a dish and a lid. The lid is thus preferably lifted off the dish when the interior of the accommodation device is opened. When the accommodation device is opened, preferably the culture medium contained in the culture medium carrier is thus made accessible at the same time. Preferably, when the interior of the accommodation device is closed, the lid is positioned back onto the dish.

The dish and the lid of the culture medium carrier can be locked to one another. In this case, the lid is preferably unlocked relative to the dish in the hermetically closed interior of the accommodation device, in particular preferably even before the accommodation device is introduced into the space of the insulator. The unlocking is preferably carried out by hand, by means of an actuating element provided therefor on the accommodation device.

After the exposure time, the lid is preferably locked to the dish in the newly hermetically closed interior of the accommodation device, in particular preferably after the accommodation device has been removed from the space of the insulator. The locking is preferably carried out by hand by means of an actuating element provided therefor on the accommodation device.

### BRIEF DESCRIPTION OF THE DRAWINGS

Preferred embodiments of the invention are described in the following with reference to the drawings, which serve illustrative purposes only and are not to be interpreted as being restrictive. In the drawings,

FIG. 1 shows a perspective view of an insulator with an accommodation device introduced therein according to an embodiment according to the invention;

FIG. 2 shows a perspective view of an accommodation device according to an embodiment according to the invention in the closed state;

FIG. 3 shows a side view of the accommodation device of FIG. 2;

FIG. 4 shows a view of the accommodation device of FIG. 2 in the fully open state, with a culture medium carrier held therein;

FIG. 5 shows a view of the accommodation device of FIG. 2 in the fully open state, without the culture medium carrier;

FIG. 6 shows a view of the accommodation device of FIG. 2 in the partially open state, with a culture medium carrier held therein;

FIG. 7 shows a central sectional view through the receptacle of the accommodation device of FIG. 2 with the culture medium carrier accommodated therein;

FIG. 8 shows an enlarged detail view of the region bordered by dashed lines in FIG. 7;

FIG. 9 shows a perspective exploded view of the receptacle of the accommodation device of FIG. 2;

FIG. 10 shows an enlarged detail view of the region bordered by dashed lines in FIG. 9;

FIG. 11 shows a central sectional view through the closure lid of the accommodation device of FIG. 2;

FIG. 12 shows an enlarged detail view of the region bordered by dashed lines in FIG. 11;

FIG. 13 shows a central sectional view through the closure lid and the receptacle of the accommodation device of FIG. 2 in the closed state, with a culture medium carrier accommodated therein;

FIG. 14 shows a perspective exploded view of parts of the closure lid of the accommodation device of FIG. 2; and

FIG. 15 shows a perspective exploded view of the locking mechanism accommodated in the closure lid of the accommodation device of FIG. 2.

### DESCRIPTION OF PREFERRED EMBODIMENTS

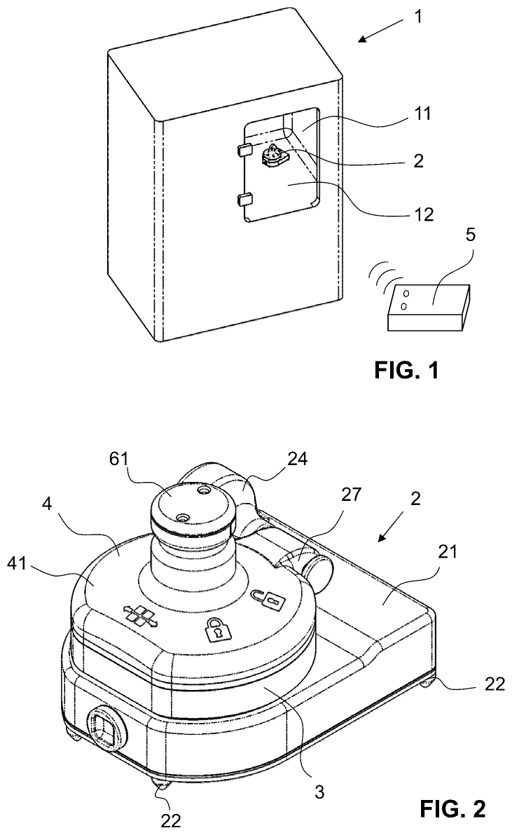

FIG. 1 shows an insulator 1 with an accommodation device 2 according to the invention introduced therein. FIGS. 2 to 15 show the accommodation device 2 according to the invention and parts thereof in different views.

As can be identified in FIG. 1, the accommodation device 2 has been introduced as a whole through an access opening 12 of the insulator 1 into a hermetically insulable space 11 of the insulator 1. A culture medium carrier which is not visible in FIG. 1 is accommodated in the accommodation device 2, the culture medium carrier being arranged in an interior of the accommodation device 2 and being hermetically insulated relative to the environment, i.e. in this case relative to the space 11 of the insulator 1. The culture medium carrier which carries a culture medium has been introduced into the previously sterilized accommodation device 2 before the latter has been introduced into the insulator 1, potentially after further sterilization. The insulator 1 can be, in particular, a gloveless insulator such as, but not exclusively, the so-called product insulator Vanrx SA25 from the firm Vanrx (part of Cytiva), Canada.

A sterilization of the space 11 can be carried out after the access opening 12 has been closed and the space 11 outwardly hermetically sealed. To this end, in particular but not exclusively, a so-called VHP (Vaporized Hydrogen Peroxide) decontamination can be carried out. The culture medium carrier is protected during this time by the accommodation device 2. When the insulator is ready for production, the accommodation device 2 in the now closed and sterilized insulator 1 can be opened from the outside wirelessly by means of a remote control 5 and the culture medium arranged on the culture medium carrier exposed thereby to the atmosphere in the space 11 of the insulator 1. The culture medium can thus be used during a certain exposure time for bio-monitoring, i.e. any microbes potentially present in the space 11 settle on the culture medium and can be detected thereby: in a further working step the culture medium can be incubated outside the insulator 1 in order to be able to identify subsequently the resulting microbe populations.

After the elapse of the exposure time the accommodation device 2 is closed again by means of the remote control 5, so that the culture medium carrier including the culture medium is hermetically insulated relative to the environment. The opening and closing of the accommodation device 2 and thus the entire handling of the culture medium carrier in the interior of the hermetically closed insulator 1 is thus carried out exclusively by means of the remote control 5. Neither a glove port nor a robot is required. An unintentional transmission of microbes in the space 11 of the insulator 1 is substantially excluded thereby. The insulator 1 can be a conventional insulator with or without a glove port.

After closing the accommodation device 2 the access opening 12 can then be opened and the accommodation device 2, which is still closed, removed as a whole from the insulator 1. Outside the insulator 1, the accommodation device 2 can then be opened and the culture medium carrier removed therefrom in order to be incubated and/or analyzed, for example. The accommodation device 2 can be used for further uses with other culture medium carriers or with new culture media.

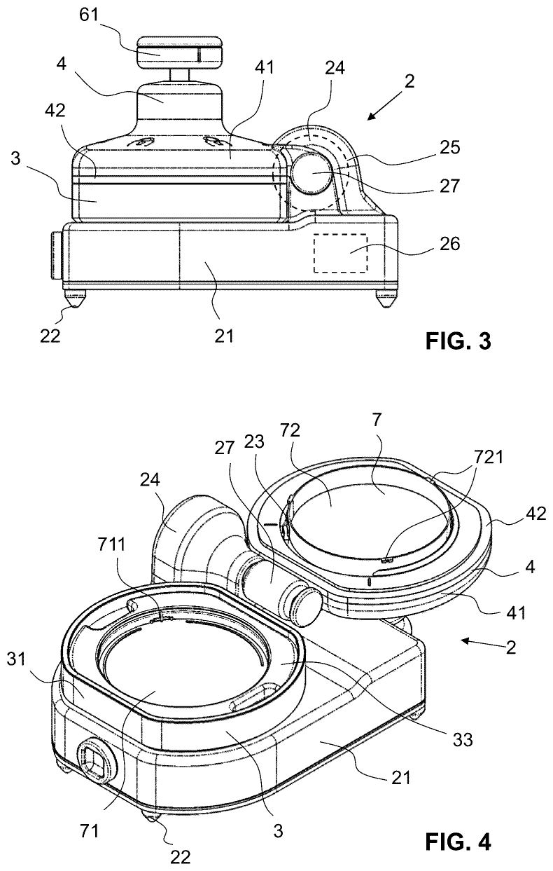

One possible embodiment of an accommodation device 2, as can be used for example in FIG. 1, is shown in FIGS. 2 to 15 in different views. As can be identified clearly in FIGS. 2 and 3, the accommodation device 2 has a base part 21 on which a receptacle 3 which can be closed by means of a closure lid 4 is arranged. To this end, the closure lid 4 is pivotable relative to the receptacle 3 about a rotary joint 27 which is arranged to the side of the receptacle 3 and the closure lid 4. The rotary joint 27 thus connects the receptacle 3 and the closure lid 4 together.

The base part 21 comprises four support feet 22, by which the accommodation device 2 can be deposited as intended on a flat surface or on which or in which a holder provided therefor can be placed.

A drive housing 24, in which a drive 25 is accommodated in the form of an electric motor (see FIG. 3), is formed by the base part 21 to the side of the rotary joint 27. The drive 25, which can be activated by means of the remote control 5, serves for opening and closing the accommodation device 2 by pivoting the closure lid 4 about the rotary joint 27. A battery 26, which is also accommodated in the base part 21, serves for supplying energy to the drive 25 as indicated in FIG. 3. The battery 26 is charged by a wireless system, for example by means of induction, so that no plugs are required therefor on the accommodation device 2. An electronic unit with a control, a storage element and/or a wireless communication device are also preferably arranged in the base part 21, but cannot all be identified in the figures.

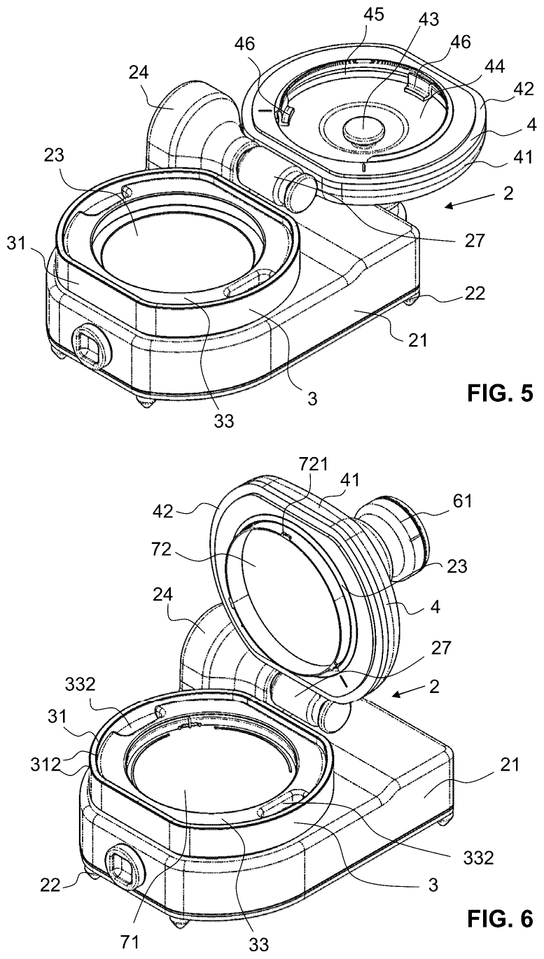

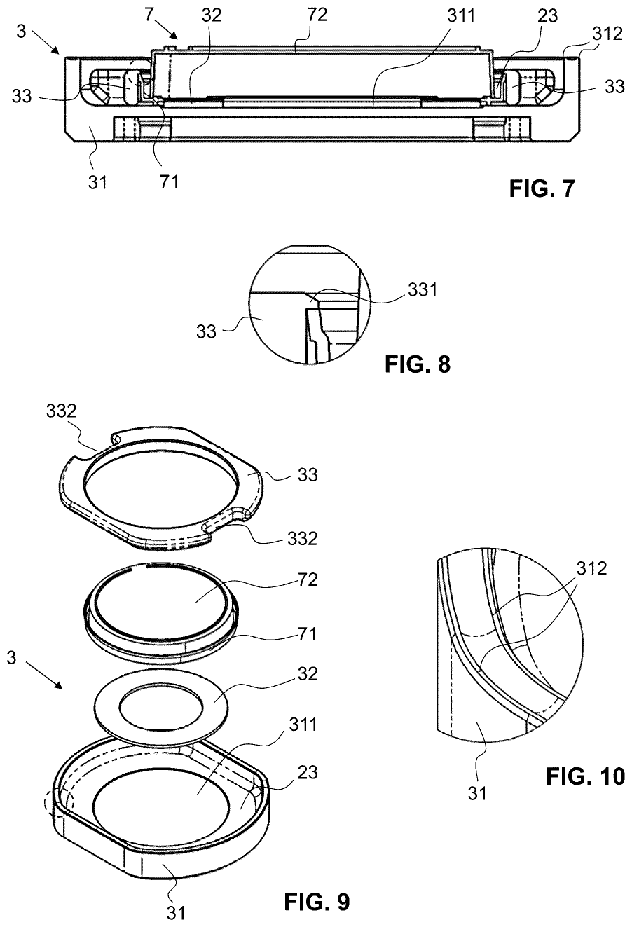

While the accommodation device 2 is shown in FIGS. 2 and 3 in the closed state, FIGS. 4 and 5 show in each case the accommodation device 2 in the fully open state. FIG. 4 shows the accommodation device 2 with, and FIG. 5 without, a culture medium carrier 7 inserted therein. The culture medium carrier 7 is configured here as a petri dish with a dish 71 and a lid 72 which can be inserted into the dish 71. The dish 71 and lid 72 can be locked to one another by means of locking elements 711 and 721. Preferably, and as is the case in the exemplary embodiment shown, the locking elements 711 and 721 together form a bayonet closure. In the present exemplary embodiment, the culture medium carrier 7 is a petri dish from the firm Merck KGaA, Germany, known under the name ICRplus Settle Plate. A cross-sectional view through the closed and locked culture medium carrier 7 is shown in FIG. 7.

The pivoting of the closure lid 4 by 180° relative to the receptacle 3, as shown in FIGS. 4 and 5, has the advantage that the culture medium accommodated in the dish 71 can thus be optimally exposed to the atmosphere in the space 11 of the insulator 1. As a result, the air can flow from all sides to the culture medium.

Preferably, it is possible to adjust the angular range about which the closure lid 4 is pivotable relative to the receptacle 3 about the rotary joint 27. Thus the closure lid 4, when opened, can also be pivoted by only 90°, for example, as shown in FIG. 6, which can be advantageous according to the situation and application.

The receptacle 3 of the accommodation device 2 is shown in more detail in FIGS. 7 to 10. The receptacle has an accommodating dish 31 with a central recess 311 which is configured with a circular area. A washer 32 which serves for supporting the dish 71 of the culture medium carrier 7 is inserted into the recess 311. A fixing ring 33 serves as a holding element for securing the dish 71 when the accommodation device 2 is opened, the fixing ring being configured with a circumferential retaining projection 331 extending radially inwardly for supporting on the dish 71 (see FIG. 7 and in particular FIG. 8). By positioning the fixing ring 33 on the dish 71 it is clamped downwardly onto the accommodating dish 31 in the closed state of the accommodation device 2 and, when the closure lid 4 is lifted and/or rotated, cannot move therewith.

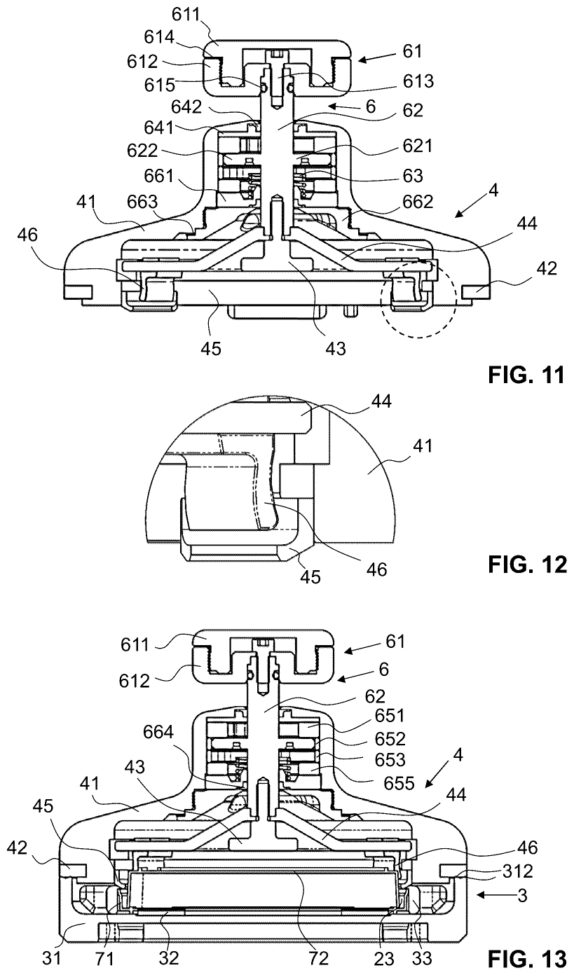

When the accommodation device 2 is closed, a further sliding ring 45, explained below (see FIGS. 11 to 13), comes to rest between the fixing ring 33 and the closure lid 4 and as a result serves for clamping the dish 71. The fixing ring 33 also serves for centering the dish 71 with its central opening after the culture medium carrier 7 is inserted into the receptacle 3. Two diametrically opposing engagement cutouts 332 which facilitate the removal of the fixing ring 33 from the accommodating dish 31 are provided on the fixing ring 33 on the outer edge. The removal of the fixing ring 33 is required when the culture medium carrier 7 is to be removed from the accommodation device 2.

The modular construction of the receptacle 3 with the accommodating dish 31, the washer 32 and fixing ring 33, in addition to the already mentioned advantages has the additional advantage that the receptacle 3 can be adapted relatively simply to other shapes, sizes and/or types of culture medium carrier 7. To this end, one or more correspondingly adapted fixing rings 33 simply have to be provided.

When the accommodation device 2 is closed, this forms a hermetically insulated interior 23 which is defined together and exclusively by the receptacle 3 and by the closure lid 4. In order to ensure a hermetic seal, the accommodating dish has two fully circumferentially configured sealing lips 312 (see FIG. 10) which are configured in each case for circumferentially bearing against a sealing ring 42 of the closure lid 4. The sealing ring 42, which can be identified for example in FIGS. 6 and 11, is inserted into a groove arranged in the region of the underside of the closure lid 4 and extends fully circumferentially along the radial outer face of the closure lid 4. Due to the duplicated design of the sealing lip 312, a particularly efficient hermetic insulation of the interior 23 is achieved and ensured.

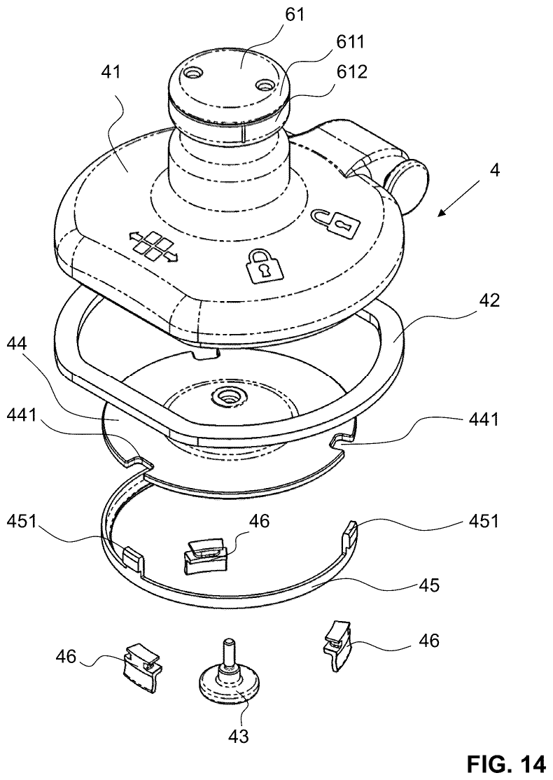

The configuration of the closure lid 4 is visible in FIGS. 11 to 15. As a basic carrying structure, the closure lid 4 comprises an envelope structure 41, as can be identified clearly for example in FIGS. 11 and 14. The already mentioned groove is configured in the lower region of the envelope structure 41 on the radial outer face, with the sealing ring 42 inserted therein.

As can also be identified in FIGS. 11 and 13, the envelope structure 41 comprises a central, vertically running through-opening in which a locking mechanism 6 is arranged. A holding plate 44 which covers the interior of the closure lid 4 downwardly and thus forms a lower end of the closure lid 4 is attached to the underside of the locking mechanism 6, as explained in more detail below. The holding plate 44 is configured to be substantially circular and has a central bulge toward the top. A through-opening which serves for fixing the main plate 44 to the actuating rod 62 is configured in the center of the bulge. The fixing takes place via a screw 43. The screw 43 is screwed through the through-opening provided on the holding plate 44 into an actuating rod 62 of the locking mechanism 6 (FIGS. 11 and 13), whereby the holding plate 44 is fastened fixedly in terms of rotation to the actuating rod 62.

On its outer face the holding plate 44 has cutouts 441 which are radially outwardly open and in which a clamping part 46 and a latching element 451 are inserted in each case. In the fully mounted state, the clamping part 46 and the latching element 451 are secured in each case thereby in the cutout 441 defined by the holding plate 44 and the envelope structure 41. The clamping part 46 is arranged radially inside the latching element 451. The latching elements 451 form in each case a part of a sliding ring 45, i.e. the latching elements 451 are connected together in one piece due to the sliding ring 45. The latching elements 451 serve for attaching the sliding ring 45 fixedly in terms of rotation to the holding plate 44. The clamping parts 46, which are otherwise not connected together, are also attached fixedly in terms of rotation to the holding plate 44 by the insertion thereof in the cutout 441.

The clamping parts 46 which extend axially downwardly toward the receptacle 3 form holding elements which are configured for the radial clamping of the lid 72 of the culture medium carrier 7. When the accommodation device 2 is closed, firstly the sliding ring 45, which does not extend fully circumferentially, slides downwardly in the axial direction along the radial outer face of the lid 72. The lid 72 is already slightly clamped thereby along a certain, not completely circumferential, region. Moreover, the lid 72 is centered by the sliding ring 45 or the closure lid 4, so that the clamping parts 46 slide radially downwardly on the outer face of the lid 72 and can clamp this lid (see FIG. 13). The lid 72 is clamped thereby fixedly in terms of rotation to the holding plate 44.

The holding plate 44 which can be identified in FIGS. 11, 13 and 14 comprises a lower bearing surface which serves for pushing down the lid 72 toward the dish 71.

The mode of operation of the locking mechanism 6 can be seen by viewing FIGS. 11, 13 and 15 together: the locking mechanism 6 has the actuating rod 62 as a central element which is already mentioned above and which extends centrally and in the vertical direction. An actuating knob 61 is fastened fixedly in terms of rotation to the actuating rod 62 at the upper end by means of a screw 613. The actuating knob 61 serves for the manual operation of the locking mechanism 6 by a user. In the present embodiment, the actuating knob 61 has an upper knob element 611 and a lower knob element 612 which are assembled so as to engage in one another such that together they form a substantially large part of the outer surface of the actuating knob 61. In the region of its radial outer edge, an outer sealing ring is clamped between the two knob elements 611 and 612 in order to prevent the penetration of particles and liquids into the interior of the actuating knob 61. An inner sealing ring 615 is arranged in a radial outer groove which is located on an upper region of the actuating rod 62 which extends into the actuating knob 61. The inner sealing ring 615 is clamped between the actuating rod 62 and the lower knob element 612 and thereby seals these two elements relative to one another.

In other embodiments, the actuating knob 61 can also be configured in one piece. Depending on the embodiment, the actuating knob 61 can also be removable from the actuating rod 62, in particular removable without the assistance of a tool. For example, the actuating knob 61 can be attached to the actuating rod 62 by means of a latching engagement. The probability of contamination can be reduced by removing the actuating knob 61 before the accommodation device 2 is introduced into the insulator 1.

The actuating rod 62 comprises a circumferential central flange 621 which is arranged in the center of the longitudinal extent thereof. A short guide projection 622 extends in each case on diametrically opposing sides outwardly from the central flange 621 in the radial direction. The two guide projections 622 serve, in combination with a guide unit 65 of the locking mechanism 6, for guiding the lid 72 when unlocking and locking the culture medium carrier 7 when the accommodation device 2 is closed. The central flange 621 and the guide projections 622 are preferably integrally formed on the vertical rod-shaped part of the actuating rod 62.

When viewed from top to bottom, the guide unit 65 comprises an upper slotted guide 651, a central slotted guide 652, a lower slotted guide 653 and an intermediate ring 655 which are connected by means of connecting pins 654 so as to prevent rotation. The elements 651, 652, 653 and 655 are all configured to be annular with a central through-opening through which the actuating rod 62 extends. In the slotted guides 651, 652 and 653 the central through-opening is configured in each case to be non-circular, however, in order to form a sliding guide which permits a rotational movement of the actuating rod only over a certain predetermined angular range and permits an axial displacement of the actuating rod only in certain predefined rotational positions.

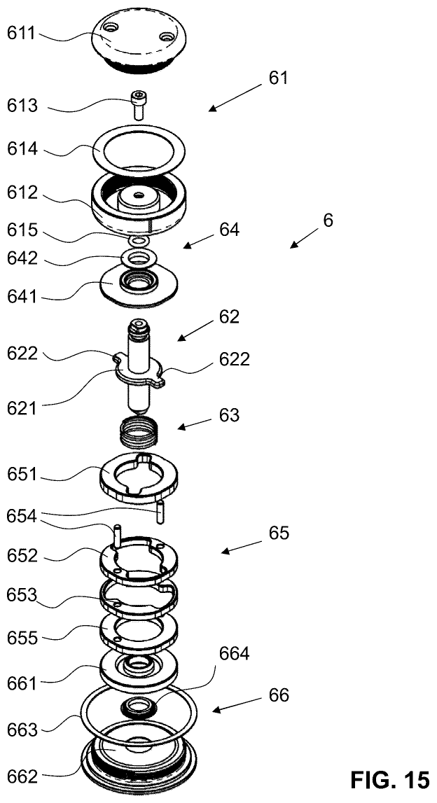

The basic position of the actuating rod 62 and thus of the locking mechanism 6 is shown in FIGS. 11, 13 and 15: the guide projections 622 of the actuating rod 62 are located level with the central slotted guide 652. When viewed from above (FIG. 15), the actuating rod 62 is rotated sufficiently far counterclockwise that the guide projections 622 bear against the slotted guide 652. In this basic position of the locking mechanism, the locked culture medium carrier 7 is generally inserted by the user into the open accommodation device 2. The situation after closing the accommodation device 2 by means of the drive 25 is shown in FIG. 13. The culture medium carrier 7 is still locked and arranged in the now hermetically insulated interior 23 of the accommodation device 2. Due to the pressure which the closure lid 4 exerts on the fixing ring 33, the dish 71 is clamped by the fixing ring 33 in the accommodating dish 31. The lid 72 is slightly clamped by the sliding ring 45.

In order to unlock the culture medium carrier 7 when the accommodation device 2 is closed, i.e. to actuate its bayonet closure between the dish 71 and lid 72, the actuating knob 61 is then pushed manually downwardly and then rotated counterclockwise by a user: the actuating rod 62 with its guide projections 622 is pushed forward thereby in the axial direction level with the lower slotted guide 653. The holding plate 44 comes to bear against the lid 72 and presses this lid toward the dish 71. At the same time, the clamping parts 46 slide downwardly along the outer face of the lid 72 and clamp the lid with a significantly greater clamping force than has been previously exerted by the sliding ring 45. The lid 72 is held thereby fixedly in terms of rotation on the holding plate 44 and thus on the actuating rod 62. The lid 72 is rotated relative to the dish 71 as a result of the subsequent rotation of the actuating rod 62 in the counterclockwise direction, as mentioned above, whereby the locking elements 711 and 721 of the bayonet closure come out of engagement. The culture medium carrier 7 is thus unlocked and the accommodation device 2 can be opened. Due to the clamping of the lid 72 by the clamping parts 46, when opened this lid is moved together with the closure lid 4 and lifted off the dish 71. The culture medium contained in the culture medium carrier 7 is thus exposed to the surrounding atmosphere.

After the accommodation device 2 is closed again by means of the drive 25, the interior 23 of the accommodation device 2 with the culture medium carrier 7 accommodated therein is again hermetically insulated from the environment. The culture medium carrier 7, however, is still unlocked, i.e. the locking elements 711 and 721 of the bayonet closure are not engaged in one another. The locking mechanism 6 is still in the same position as when the accommodation device 2 is opened, i.e. the guide projections 622 of the actuating rod 62 are arranged level with the lower slotted guide 653.

In order to lock the culture medium carrier 7 when the accommodation device 2 is closed, i.e. to lock the dish 71 and the lid 72 together by means of the bayonet closure, the actuating knob 61 is manually rotated clockwise and then pulled upwardly by the user: due to the clockwise rotation the lid 72 which continues to be secured by the clamping parts 46 is rotated therewith, whereby the locking elements 711 and 721 of the dish 71 and the lid 72 are again brought into engagement with one another and thereby lock the culture medium carrier 7. When the actuating knob 61 is subsequently pulled back axially upwardly, the clamping parts 46 are pulled back relative to the lid 72 since this lid is now held on the dish 71 due to the locked bayonet closure. The lid 72 is thus no longer clamped by the clamping parts 46 and the locking mechanism 6 is again in its basic position shown in FIGS. 11, 13 and 15.

The accommodation device 2 can then be opened again by means of the drive 25, wherein the lid 72 remains on the dish 71 when opened. This is because the lid 72 is firstly held via the bayonet closure on the dish 71 and secondly is no longer clamped by the clamping parts 46.

A further position of the locking mechanism 6, which is denoted hereinafter as the dismantled position, is permitted by the above slotted guide 651. To this end, the actuating knob 61 has to be rotated clockwise by the user from the basic position and then pulled upwardly: since the upper slotted guide 651 has a radial inner surface which specifically replicates in a complementary manner the central flange 621 and the guide projections 622, any further rotation of the actuating rod 62 is made impossible in the dismantled position. In the dismantled position, the actuating rod 62 together with the holding plate 44 attached thereto is therefore pulled back further upwardly into the envelope structure 41 relative to the situation shown in FIGS. 11 and 13. The dismantled position serves for simpler dismantling of the closure lid 4, for example for cleaning and/or sterilizing purposes. The locking mechanism 6 can be brought back easily into the basic position by pushing down the actuating knob 61 from the dismantled position and then rotating counterclockwise.

Relative to the locking mechanism 6, markings are preferably applied to the outer face of the closure lid 4 and the actuating knob 61, the markings enabling a user to identify the dismantled position, the basic position and the position in which the culture medium carrier 7 is unlocked in the interior of the accommodation device 2. Corresponding markings can be identified in FIGS. 2 and 14.

As can also be identified in FIGS. 11, 13 and 15, the locking mechanism 6 can have a compression spring 63 in order to apply a force to the actuating rod 62 axially in the direction of the basic position or dismantled position. The compression spring 63 can be configured, in particular, as a spiral spring which extends around the actuating rod 62 and in the axial direction, on the one hand, bears against the central flange 621 of the actuating rod 62 and, on the other hand, against a spring stop disk 661 which is stationary relative to the envelope structure 41.

The spring stop disk 661 is part of a lower fastening unit 66, which also includes a lower fastening disk 662. The lower fastening disk 662 is screwed with an external thread into an internal thread of the envelope structure 41 and thereby forms a lower end of the locking mechanism 6. For sealing the locking mechanism 6 and thus the closure lid 4 downwardly, a lower outer sealing ring 663 is provided, which is arranged circumferentially between the lower fastening disk 662 and the envelope structure 41 and seals them relative to one another. A lower inner sealing ring 664 seals the lower fastening disk 662 in the region of its central through-opening circumferentially relative to the actuating rod 62. The spring stop disk 661, which also has a central through-opening for passing through the actuating rod 62, is positioned on the upper face of the lower fastening disk 662.

At the top, the locking mechanism 6 comprises an upper fastening unit 64 with an upper fastening disk 641. The upper fastening disk 641 is arranged above the upper slotted guide 651. An upper sealing ring 642 is clamped between the envelope structure 41 and the upper fastening disk 641 in order to seal the envelope structure 41 and the upper fastening disk 641 relative to the actuating rod 62. The actuating rod 62 extends through a central through-opening of the upper fastening disk 641.

The present invention is not limited to the embodiments shown in the figures and the description, and a variety of modifications is possible. Thus, the culture medium carrier, for example, does not necessarily have to comprise a lid which can be locked to a dish. The culture medium carrier could be formed just as well by a flat plate. Accordingly, the accommodation device does not necessarily have to comprise a locking mechanism, even when this is preferred. The opening of the accommodation device also does not necessarily have to take place by pivoting a closure lid. In certain embodiments, in order to be opened for example, the closure lid could also be moved away from the receptacle or displaced relative to a window opening in a translational manner. If a locking mechanism is present, this can also be operated by means of a motorized drive rather than manually. Various of the described individual elements are also not necessarily required, and in other embodiments could also be completely dispensed with or configured differently. The guide slots 651, 653 and 654 and potentially the upper fastening disk 641, the spring stop disk 661 and/or the lower fastening disk 662 could be formed together by a one-piece element, for example, whereby the connecting pins 654 could be dispensed with. Further alterations are possible.

LIST OF REFERENCE SIGNS

| | |
|---|---|
| 1 | Insulator |
| 11 | Hermetically insulable space |
| 12 | Access opening |
| 2 | Accommodation device |
| 21 | Base part |
| 22 | Support foot |
| 23 | Interior |
| 24 | Drive housing |
| 25 | Drive |
| 26 | Battery |
| 27 | Rotary joint |
| 3 | Receptacle |
| 31 | Accommodating dish |
| 311 | Recess |
| 312 | Sealing lip |
| 32 | Washer |
| 33 | Fixing ring |
| 331 | Retaining projection |
| 332 | Engagement cutout |
| 4 | Closure lid |
| 41 | Envelope structure |
| 42 | Sealing ring |
| 43 | Screw |
| 44 | Holding plate |
| 441 | Cutout |
| 45 | Sliding ring |
| 451 | Latching element |
| 46 | Clamping part |
| 5 | Remote control |
| 6 | Locking mechanism |
| 61 | Actuating knob |
| 611 | Upper knob element |
| 612 | Lower knob element |
| 613 | Screw |
| 614 | Outer sealing ring |
| 615 | Inner sealing ring |
| 62 | Actuating rod |
| 621 | Central flange |
| 622 | Guide projection |
| 63 | Compression spring |
| 64 | Upper fastening unit |
| 641 | Upper fastening disk |
| 642 | Upper sealing ring |
| 65 | Guide unit |
| 651 | Upper slotted guide |
| 652 | Central slotted guide |
| 653 | Lower slotted guide |
| 654 | Connecting pin |
| 655 | Intermediate ring |
| 66 | Lower fastening unit |
| 661 | Spring stop disk |
| 662 | Lower fastening disk |
| 663 | Lower outer sealing ring |
| 664 | Lower inner sealing ring |
| 7 | Culture medium carrier |
| 71 | Dish |
| 711 | Locking element |
| 72 | Lid |
| 721 | Locking element |

1. An accommodation device for a culture medium carrier, comprising
a hermetically closable interior for accommodation of the culture medium carrier; and
a wirelessly remote-controllable drive for opening and closing the accommodation device in order respectively to provide access to the culture medium carrier or to hermetically close the interior with the culture medium carrier accommodated therein;
wherein the accommodation device as a whole can be introduced into and removed from a hermetically insulable space in an insulator.

2. The accommodation device as claimed in claim 1, also comprising at least one energy storage element for supplying electrical energy to the drive.

3. The accommodation device as claimed in claim 1, wherein the accommodation device is configured, when opened, to lift a lid of the culture medium carrier off a dish of the culture medium carrier and, when closed, to position the lid back onto the dish.

4. The accommodation device as claimed in claim 3, comprising a first holding element for securing the lid and a second holding element for securing the dish when the accommodation device is opened and closed.

5. The accommodation device as claimed in claim 3, wherein the lid can be locked to the dish and wherein the accommodation device is configured to unlock and to lock the culture medium carrier which is accommodated in the hermetically closed interior.

6. The accommodation device as claimed in claim 5, comprising a locking mechanism for unlocking and locking a culture medium carrier which comprises a threaded or bayonet closure.

7. The accommodation device as claimed in claim 6, wherein the locking mechanism comprises a first holding element for securing the lid and wherein the first holding element can be moved from an initial position into a pushed-down position and from the pushed-down position into a pushed-down rotated position, in order to lock a culture medium carrier which comprises a bayonet closure.

8. The accommodation device as claimed in claim 1, comprising a receptacle and a closure lid which together define the interior, wherein the closure lid can be pivoted for opening and closing the interior over an angular range of at least 45° about a rotary joint.

9. The accommodation device as claimed in claim 8, wherein it is possible to select the angular range over which the closure lid can be pivoted about the rotary joint.

10. The accommodation device as claimed in claim 1, comprising a fully circumferential, at least duplicated, sealing lip in order to seal hermetically the interior in the closed state.

11. A method for identifying microbes in the atmosphere of an insulator comprising at least the following steps:
introducing an accommodation device into a space of the insulator, wherein the accommodation device comprises a hermetically closed interior in which a culture medium carrier, is accommodated;
hermetically closing the space of the insulator with the accommodation device accommodated as a whole therein; and
opening the interior of the accommodation device by means of a remote control which is arranged outside the hermetically closed space of the insulator.

12. The method as claimed in claim 11, also comprising the following steps:
hermetically closing the interior of the accommodation device by means of the remote control after a certain exposure time, during which a culture medium carried by the culture medium carrier has been exposed to the atmosphere of the hermetically closed space of the insulator;
opening the space of the insulator; and
removing the accommodation device as a whole from the space of the insulator.

13. The method as claimed in claim 11, wherein the culture medium carrier comprises a dish and a lid and wherein the lid is lifted off the dish when the interior of the accommodation device is opened.

14. The method as claimed in claim 13, wherein the dish and the lid of the culture medium carrier can be locked to one another, and wherein the lid is unlocked relative to the dish in the hermetically closed interior of the accommodation device.

15. The method as claimed in claim 14, wherein the lid is unlocked relative to the dish in the hermetically closed interior of the accommodation device even before the accommodation device is introduced into the space of the insulator.

16. The method as claimed in claim 11, wherein the culture medium carrier is a petri dish.

17. The method as claimed in claim 11, wherein the accommodation device comprises a wirelessly remote-controllable drive for opening and closing the accommodation device in order respectively to provide access to the culture medium carrier or to hermetically close the interior with the culture medium carrier accommodated therein.

18. The accommodation device as claimed in claim 1, wherein the culture medium carrier is a petri dish.
